(19) United States
(12) Patent Application Publication
(10) Pub. No.: US 2007/0147628 A1
(43) Pub. Date: Jun. 28, 2007

(54) Hearing protection device

(76) Inventor: Randy E. Benway, Horicon, WI (US)

Correspondence Address: RYAN KROMHOLZ & MANION, S.C., POST OFFICE BOX 26618, MILWAUKEE, WI, 53226, US

(21) Appl. No.: 11/633,717

(22) Filed: Dec. 5, 2006

Related U.S. Application Data
(63) Continuation-in-part of application No. 10/816,458, filed on Apr. 1, 2004, now abandoned.

(51) Int. Cl. A61F 11/06

(52) U.S. Cl. 381/71.2

(57) ABSTRACT

A hearing protection unit is preferably coupled with a conventional hat, such as a baseball cap, and preferred routing channels extend proximal to an ear of a wearer of the hat. The hearing protection unit amplifies all sounds and delivers a signal to a speaker, which generates sound to be aurally communicated to a wearer's ears. Upon sensing a predetermined threshold input signal, the speaker signal is modified, thereby allowing controlled suppression of damaging noises.

### RELATED APPLICATION

This application is a continuation-in-part of U.S. application Ser. No. 10/816,458, filed Apr. 1, 2004.

### BACKGROUND OF THE INVENTION

This invention relates generally to hearing protection and more particularly to hearing protection suited for loud peak noise environments such as firearms ranges and construction sites.

To safeguard against permanent hearing loss, hearing protection is essential while firing a firearm or experiencing any loud sound such as from a firing range or operating equipment. Hearing experts normally recommend that hearing protection be worn whenever sound levels regularly exceed 85 decibels (dB) for extended periods. Noise levels from discharging a firearm can range from 120-170 dB. The noise emission from just one gun blast can cause permanent hearing damage. Hunters and shooters generally know they should wear hearing protection but sometimes don't because of the desire to hear conversation, sounds of the woods and birds flushing. Many shooters do not wear hearing protection because they just don't find it comfortable.

Hearing protectors fall generally into two categories: active and passive. Passive devices simply block noise from entering the ear canal. Common foam ear plugs and most over-the-ear muffs are passive. In addition to blocking noise from reaching the ear, some active hearing protection uses external microphones to sample sound, amplifies the signal, and sends it to speakers where the user can hear the external sounds at a safe level. Noise canceling devices also fall into the active category. These devices use microphones to actively monitor what noise is getting past the protector. An electronic processor creates a correction signal that is amplified and emitted into the device. The correction signal then acts to negate the unwanted noise. Noise canceling protectors are effective when used in constant noise environments but do not adequately handle sudden loud sounds, such as a gun shot.

Electronic hearing protection exists for the shooting industry in three basic types: muffs, behind-the-ear (BTE), and in-the-ear (ITE). Ear muff styles are worn over the ears during shooting. Some active type muffs incorporate eternal microphones that sample sound and send the signals to circuitry that amplifies the signal and sends it to speakers. Separate volume controls are typically on each earpiece. Muffs are very effective and generally inexpensive, but muffs are large in size. Size is the biggest problem for a person discharging a firearm as many times the stock of the firearm contacts the muffs upon mounting the shotgun to the shoulder. Muffs can also be quite hot to wear. Proper fit is a big factor in the effectiveness of any type of hearing protection. This proper fit is often interfered by shooting or safety glasses.

Separate electronic ITE plugs are also available. They too have volume controls on each plug, which requires a user to manually balance the sound levels. While satisfying desirable size characteristics, the cost of this type ear protection is often considerable. The small controls on these devices make adjusting and balancing the volume difficult.

Behind-the-ear active hearing protectors are also available. Usually, a small module connects to an ear plug via a nylon tube. The tube routes from the ear plug, over the ear, to a module nested behind the ear. Housing the electronics behind the ear provides more room for circuitry, which generally lowers cost below the cost of comparable ITE units. Although effective, BTE devices, like muffs, interfere with eyeglass use and require separate controls to operate left and right channel volume.

Therefore, the firearms field could benefit from a device that provides the following: proper hearing protection from loud gun discharges, the amplification of desired sounds and suppression of others, and a comfortable fit for extended use while avoiding interfering contact with a firearm. Additionally, attaching such a device to a carrier such as a hat further provides for larger and additional battery storage capability.

### SUMMARY OF THE INVENTION

A hearing protector is disclosed that amplifies desirable sounds, such as voices, but suppresses loud sounds such as shotgun discharges. Furthermore, the hearing protector does not interfere with the positioning of a shouldered firearm.

The present invention is a design where a microphone, at least one speaker, and other circuitry is packaged in a housing that could be affixed to, for instance, the underside of a cap brim, thereby providing a method of a greatly reduced cost as compared to the prior art. Alternatively, rather than having the speaker or speakers mounted in the housing, the speaker or speakers are selectively mountable close to the ears of the wearer.

If speakers are mounted remote from the ears, tubing from the module may be routed through the hat, exiting the sides, where it is connected to earphones, much like a stethoscope. Mono or stereo models can be provided. Alternatively, if speakers are selectively mountable close to the ears of the wearer, electrical wiring is used to transmit representative electrical signals from the module to the speakers, which can be selectively engaged with a wearer's ear.

The hearing protection unit may be coupled with a conventional hat, such as a baseball cap. Additionally, the hearing protection unit can be coupled with hard hats such as those used in industrial applications.

### BRIEF DESCRIPTION OF THE DRAWINGS

FIG. 1 is a block diagram of a first preferred electrical circuit according the present invention.

FIG. 1a is an electrical schematic of a preferred audio pick-up incorporated into the first preferred electrical circuit of the present invention.

FIG. 1b is an electrical schematic of a preferred volume controller incorporated into the first preferred electrical circuit of the present invention.

FIG. 1c is an electrical schematic of a preferred amplifier incorporated into the first preferred electrical circuit of the present invention.

FIG. 1d is an electrical schematic of a preferred monitor incorporated into the first preferred electrical circuit of the present invention.

FIG. 1e is an electrical schematic of a preferred power supply incorporated into the first preferred electrical circuit of the present invention.

FIG. 1f is an electrical schematic of a preferred audio compression circuit incorporated into the first preferred electrical circuit of the present invention.

FIG. 2 is a top perspective view of a preferred embodiment of a hearing protection device of the present invention.

FIG. 3 is a bottom perspective view of a preferred embodiment of a hearing protection device of the present invention.

FIG. 4 is a top perspective view of a second preferred embodiment of a hearing protection device of the present invention.

FIG. 5 is a bottom perspective view of a third preferred embodiment of a hearing protection device of the present invention.

FIG. 6 is a top perspective view of the third preferred embodiment in use while disposed on a user's head.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment is hereinafter described, the details may be changed without departing from the invention, which is defined by the claims.

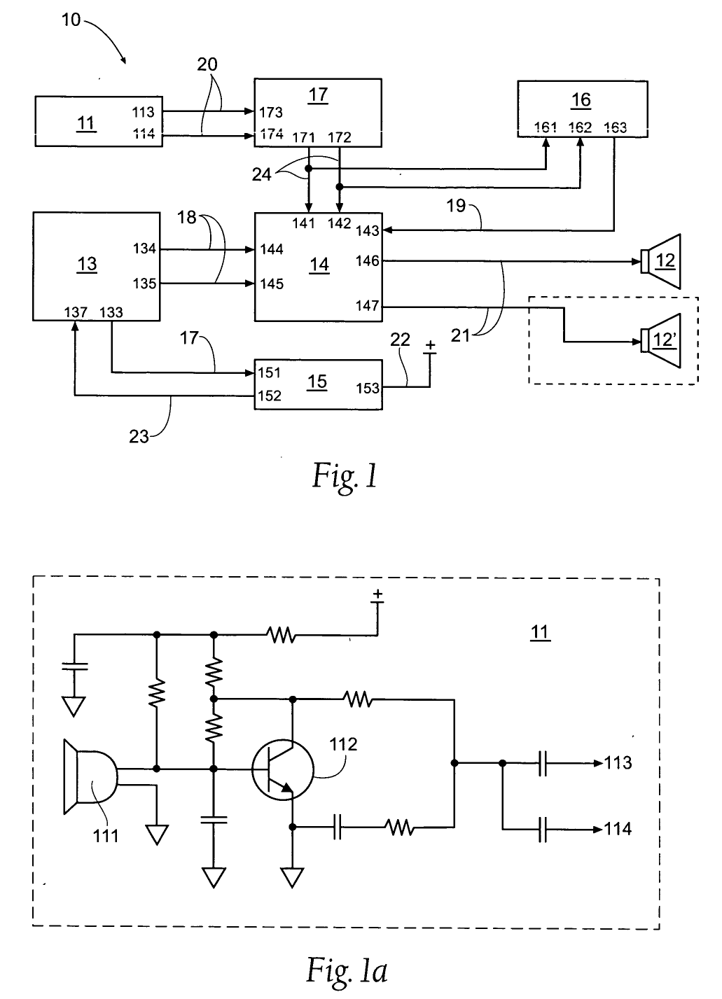

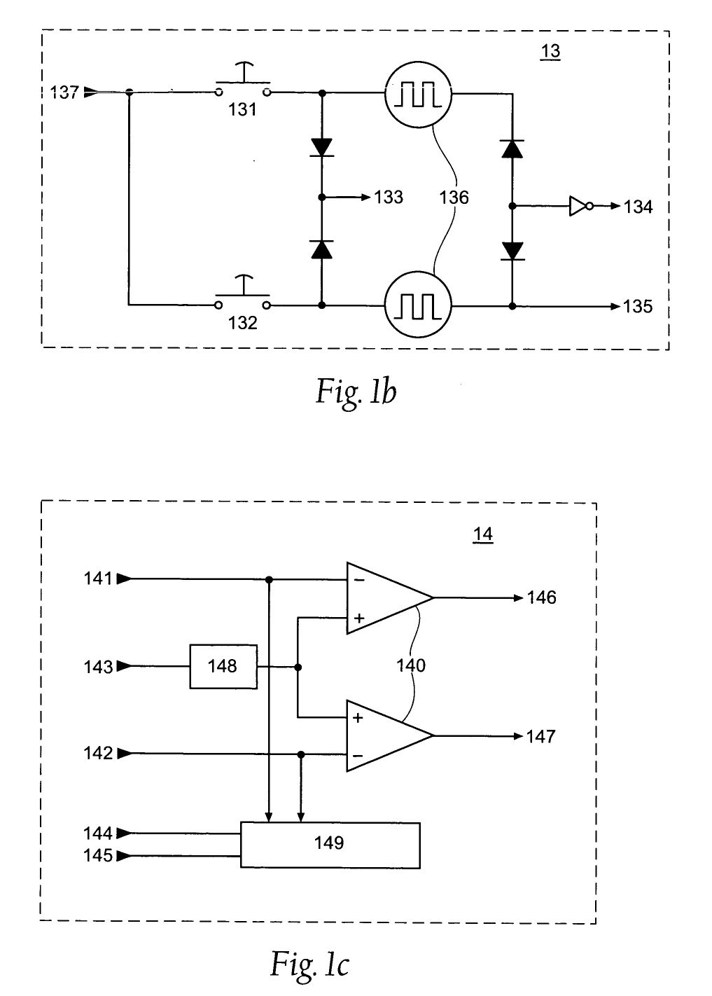

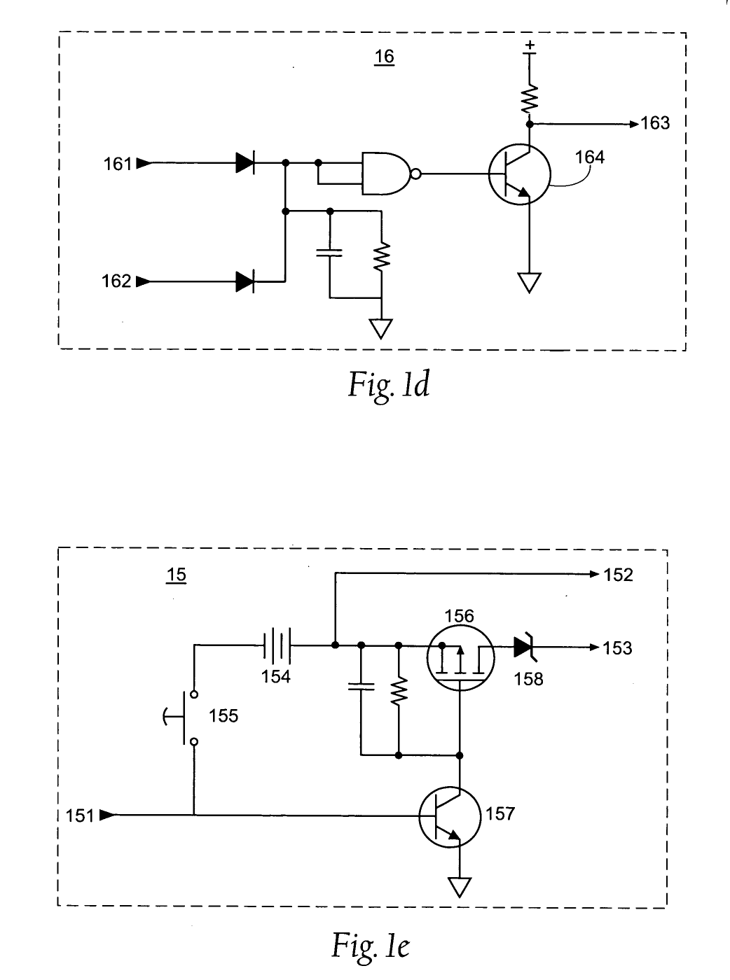

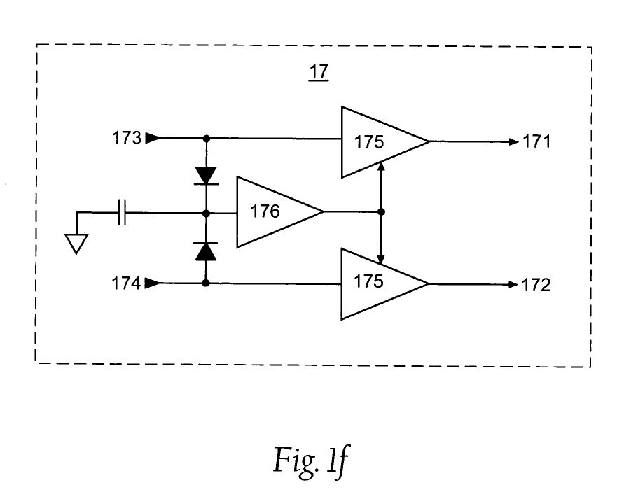

FIG. 1 depicts a block diagram of an electronic circuit 10 embodiment to be used with the hearing protection device of the present invention. Generally, the circuit 10 has an audio pick-up 11 electrically coupled to at least one speaker 12. Optionally, a second speaker 12′ may be added to the circuit 10. More specifically, the circuit 10 comprises a volume controller 13, an amplifier 14, a power supply 15, the audio pick-up 11, an audio compressor 17, a monitor 16, and the speaker 12. The power supply 15 provides the system operating voltage 22 and a constant battery voltage 23 to the volume controller 13. The volume controller 13 provides a power-on signal 17 to the power supply 15 and also provides volume control 18 to the amplifier 14 to increase or decrease the level of the signals 21 provided to the speaker 12. The audio pick-up 11 provides electrical signals 20, which are representative of audio signals received by the pick-up 11, to the compressor 17. The compressor 17 provides compressed audio signals 24 to the amplifier 14. The amplifier 14 provides controlled electrical outputs 21 to the speakers 12, 12′ which are representative of the signals 24 received from the compressor 17 based on the configuration of the amplifier 14 as manipulated by the volume controller 13.

During times when ambient sound is at desired levels, the amplifier 14 outputs signals 21 at a selected volume to the speaker 12. However, it is desirable to suppress loud noises that may otherwise harm the wearer's ear. To prevent harm, the circuit 10 is provided with a means for modifying the signals 21 provided to the speaker 12. The circuit 10 is also provided with a monitor 16, which preferably controls the means for modifying the signals 21. The means for modifying, as depicted, is preferably an amplifier shut down control input 143. The monitor 16 samples the signals 24 presented to the amplifier 14 signal inputs 141,142. Upon detecting undesirable signal levels, the monitor 16 communicates a signal 19 to the means for modifying 143 to deactivate the outputs 146,147 of the amplifier 14. Upon return of acceptable ambient noise levels, or after a predetermined period of time, or both, the signal 19 is deactivated and the amplifier 14 is allowed to once again provide volume-controlled electrical outputs 21 to the speakers 12,12′. While the compressor 17 is not needed to achieve amplification deactivation, the compressor 17 is desirable to provide the relatively constant signal level to the amplifier 14. Without the compressor 17, the outputs 113,114 of the audio pick-up 11 may be directly coupled to the signal inputs 141,142 of the amplifier 14, to which the monitor inputs 161,162 would remain coupled.

As seen in FIG. 1a, and also with reference to FIG. 1, the audio pick-up 11 preferably comprises a microphone 111 and appropriate bias circuitry including a low noise, high gain transistor amplifier 112. In this embodiment, the audio pick-up 11 output is replicated 113 to interface to both inputs 173,174 of the compressor 17. In this manner, a mono audio signal is eventually supplied to a single speaker 12, or both speakers 12, 12′. Optionally, a second microphone could be included in the audio pick-up 11. Inclusion of a second microphone enables two-channel stereo sound. If a second microphone is included, a similar bias circuit to that of the first microphone 111 is preferably used. Further, in an embodiment using two microphones, instead of capacitively coupling both outputs 113,114 together, one output 114 is capacitively coupled to the second microphone bias circuit in a manner similar to the coupling of the other output 113 to the first microphone 111 bias circuit.

Referring now to FIG. 1b, and also with reference to FIG. 1, an electrical schematic of an embodiment of the volume controller 13 is shown. Generally, the volume controller 13 preferably controls activation of the circuit 10 and volume control of the amplifier 14 outputs. The preferred volume controller 13 comprises a volume increase switch 131, a volume decrease switch 132, a power-on signal output 133, a clock signal output 134, a volume increase/decrease signal output 15, a pair of oscillators 136, and a battery power input 137. Although various interfaces could be used, the volume switches 131,132 are preferably push-button, normally open switches, each having one pole connected to the battery power input 137 and the other pole connected to one of the provided oscillators 136. Depression of either the volume increase switch 131 or the volume decrease switch 132 activates the power-on output 133 to indicate to the power supply 15 that the circuit 10 requires system power 22. In addition to sending the power-on signal 133, the depression of the volume increase switch 131 activates one of the oscillators 136, which sets the volume increase/decrease signal 135 to an appropriate volume increase logic level and causes an oscillating signal on the clock output 134. Similarly, depression of the volume decrease switch 132 activates the other of the oscillators 136, which sets the volume increase/decrease signal 135 to the appropriate volume decrease logic level and causes an oscillating signal on the clock output 134. Release of either depressed switch 131 or 132 causes the clock output 134 to cease oscillation, thereby preventing further volume change. Furthermore, continued depression of either switch 131 or 132 causes an increase or decrease in volume, respectively, with each oscillation, or predetermined number of oscillations, of the active oscillator 136. The volume controller 13 described to this point would be most applicable in a circuit 10 that incorporates a digital volume control for the amplifier 15. Alternatively, the volume controller 13 could consist of a simple potentiometer that may include an OFF position. In This instance, the volume controller would still control power supply to the circuit 10, but the variable resistance of the potentiometer would vary the biased gain of preferably an analog volume controlled amplifier 14.

Turning to FIG. 1c, and also with reference to FIG. 1, a preferred amplifier 14 is shown. One embodiment of the invention utilizes a standard headphone amplifier 14, which basically comprises two op-amps 140, wherein the two inputs 141,142 of the amplifier 14 are tied to op-amp 140 inverting inputs through appropriate bias circuitry. The amplifier 14 may be an integrated circuit, such as an LM4811 available from National Semiconductor Corporation of Santa Clara, Calif., or constructed of discrete electronic components. The outputs 146,147 of the amplifier 14 are electrically coupled to the speakers 12,12′. If only one speaker 12 is used, only one output 146 or 147 may be desired. Furthermore, the amplifier 14 preferably includes a digital volume controller 149, which includes as inputs a clock 144 and a signal 145 representative of volume increase or decrease. Preferably, the digital volume controller 149 provides a plurality of digital steps of amplification. While any number of combinations could be employed, it is preferable to have sixteen digital steps, wherein each step causes a 3 dB change in the volume. Generally, the digital volume controller 149 controls the gain of the op-amps 140. The amplifier 14 further includes shutdown circuitry 148, which is tied to a shutdown input 143. When the shutdown input 143 is activated, the shutdown circuitry 148 biases the op-amps amps 140 such that the amplifier outputs 146,147 are deactivated. Alternatively, if an analog amplifier 14 is used and the amplifier 14 is not provided with shutdown circuitry 148, the monitor 16, as described below, could effectively prevent amplification in a number of ways.

An electrical schematic of an embodiment of the monitor 16 is shown in FIG. 1d. The monitor 16 comprises preferably the same number of inputs 161,162 as the number of amplifier 14 inputs employed, a shutdown signal output 163, and a switch 164. If any input 161 or 162 exceeds a predetermined level, the switch 164, which is normally turned on, will turn off, thereby causing the shutdown signal output 163 to activate. The shutdown signal output 163 is preferably connected to the means for modifying the signals 21 presented to the speakers 12,12′, in this case a shutdown signal input 143 of the amplifier 14. Activation of the shutdown control input 143 of the amplifier 14 prevents amplification and passage of an amplified signal to the speakers 12,12′.

As described above, the means for modifying the signals 21 presented to the speakers 12,12′ is the shutdown input 143 of the amplifier 14, which ceases amplification by deactivating the amplifier 14. However, the signals 21 could be modified in other ways. For instance, the monitor 16 could prevent amplification by interrupting the inputs to the amplifier 14 after sensing a predetermined signal 24 level, in which case the means for modifying may be a simple transistor switching circuit. Additionally, rather than ceasing amplification completely, the means for modifying the signals 21 could reduce the gain of the amplifier 14. To employ this functionality, a volume override circuit may be controlled by the monitor 16. Finally, the monitor 16 may simply disconnect or interrupt the signals 21 presented to the speaker 12. This arrangement may be accomplished by methods well known in the art for switching signals.

FIG. 1e provides an electrical schematic of an embodiment of the power supply 15. The power supply includes a battery 154, a power-off switch 155, a power-on latch 157, a power switch 156, a power-on signal input 151, a battery power output 152, and a system voltage output 153. The battery 154 is provided to power the circuit 10. Generally, prior to circuit activation by the volume controller 13, the battery 154 is disconnected from the circuit 10, except the volume controller 13, because the power-on latch 157 is deactivated and the power switch 156 is turned off. The battery power output 152, however, is active and is connected to the volume controller 13. When the volume controller 13 activates the power-on signal input 151, which is preferably done by the user depressing either volume switch, the power-on latch 157 is turned on. In turn, the power switch 156 turns on, thereby providing the battery 154 voltage to the rest of the circuit 10 via the system voltage output 153. To conserve battery 154 power, the circuit 10 may be deactivated. While this may be accomplished in a variety of ways, preferably a power-off switch 155 is used. The power-off switch 155 is a push-button, normally open switch. Depression of the switch 155 causes the power-on latch 157 to deactivate, thereby turning off the power switch 156. Thus, battery 154 power is removed, once again, from the entire circuit 10, except the volume controller 13.

FIG. 1f provides an electrical schematic of an embodiment of the compressor 17. The compressor 17 comprises outputs 171,172, inputs 173,174, signal amplifiers 175, and a gain controller 176. Generally, as is understood in the art, the compressor 17 provides relatively constant outputs 171,172, within a predetermined range. These outputs 171,172 are fed to the amplifier 14 and are sampled by the monitor 16. The compressor inputs 173,174 are coupled to the outputs 113,114 of the audio pick-up 11. To accomplish the relatively constant output over a predetermined range, the compressor 17 gain controller 176 modifies the gain of the amplifiers 175 in a relationship that is inversely proportional to the signal level provided at the inputs 173,174. That is, for weaker input levels, the gain controller 176 will increase the gain of the amplifiers 175. Conversely, the gain controller 176 will decrease the gain of the amplifiers 175 when the levels of the inputs 173,174 increase. If the outputs 171,172 increase beyond a threshold level, the signals 21 provided to the speaker 12 will be modified, as described above.

It is to be appreciated that the sound generated by the speaker 12 is to be conveyed to at least one ear of the wearer of the device. Such transmission of sound can be accomplished in a variety of ways, two of which are herein described.

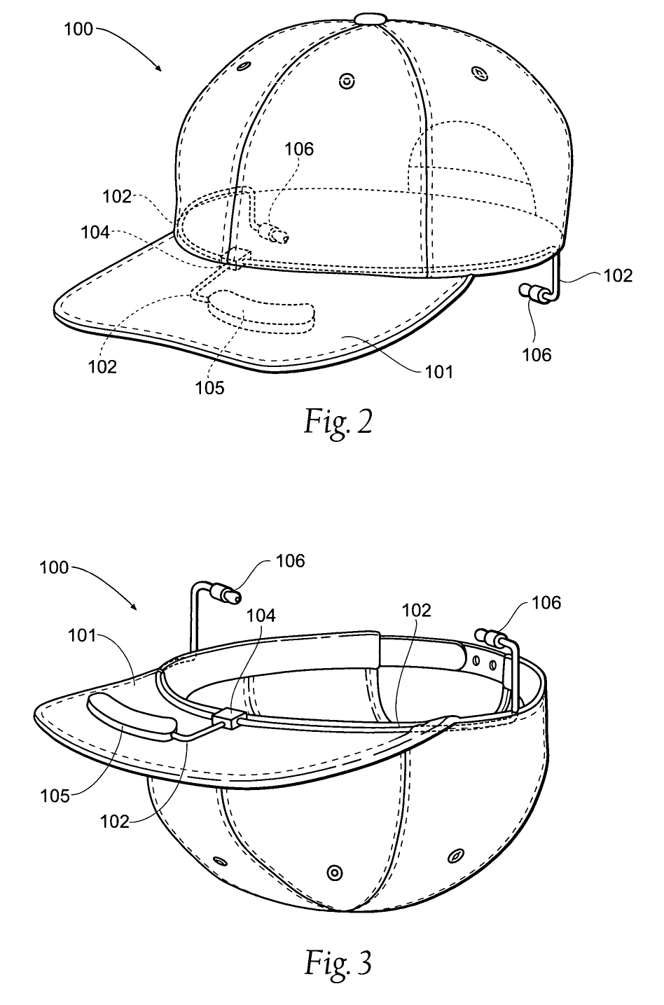

Referring to FIGS. 2-3, an embodiment 100 of a hearing protection device of the present invention is shown, wherein the speaker 12 is placed distal the ear of the wearer. For example, the speaker 12 may be mounted on the same substrate as the circuit 10. Preferably, the circuit 10 is packaged in a single housing 105 that may be affixed to an underside of a brim of a cap 101. Where the speaker 12 is distal the ear of the wearer, it is preferable to use tubing 102 to convey the sound created by the speaker 12 to the ear of the wearer. That is, the speaker 12 is aurally coupled to the ear of the wearer via the tubing 102 and earpieces 106. If desirable to convey sound to both ears of the wearer, multiple lengths of tubing 102 can be used.

The tubing 102 extends from the housing 105 and is routed through the cap 101, exiting the sides, where it is connected to the earpieces 106. As described above, mono or stereo models can be provided. Preferably, the tubing 102 is coupled to a tee-fitting 104 offering a split for additional tubing 102. An example of the tubing 102 used is 1/16″ inside diameter tubing. Proximate the ears of the wearer, the tubing 102 is either formed or pieced in a manner that guides the tubing 102 down from the cap 101 and towards the ears of the wearer and connected to earpieces 106.

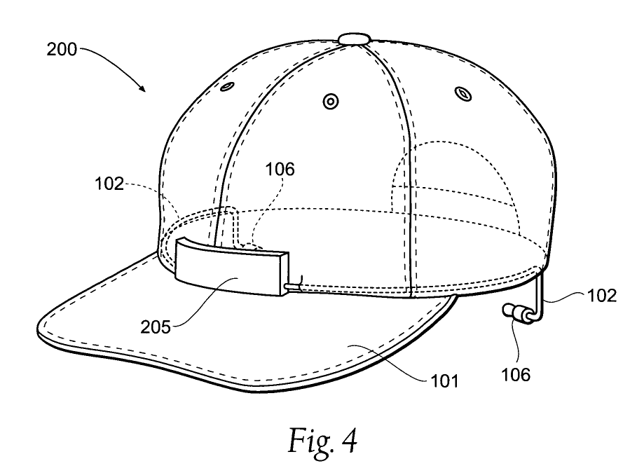

Referring now to FIG. 4 a top perspective view of a second preferred embodiment of a hearing protection device of the present invention is shown. Like the first embodiment of FIGS. 2 and 3, this embodiment has a speaker placed distal the ear of a wearer. In this embodiment, rather than place a housing under the brim of a hat, a microphone/speaker assembly 205 is coupled on the outside of the cap 101, preferably on the front of the cap 101, where designs or slogans may be emblazoned on the assembly 205. In this arrangement, tubing 102 can either be routed through the cap 101, as shown, and then in the area proximal to the ear, or routed about the hat 101 and routed rearward (not shown). As will be appreciated, the microphone/speaker assembly 205 could also be coupled to different portions of the cap 101, such as the top of the brim, or the back, in accordance with manufacturer preference.

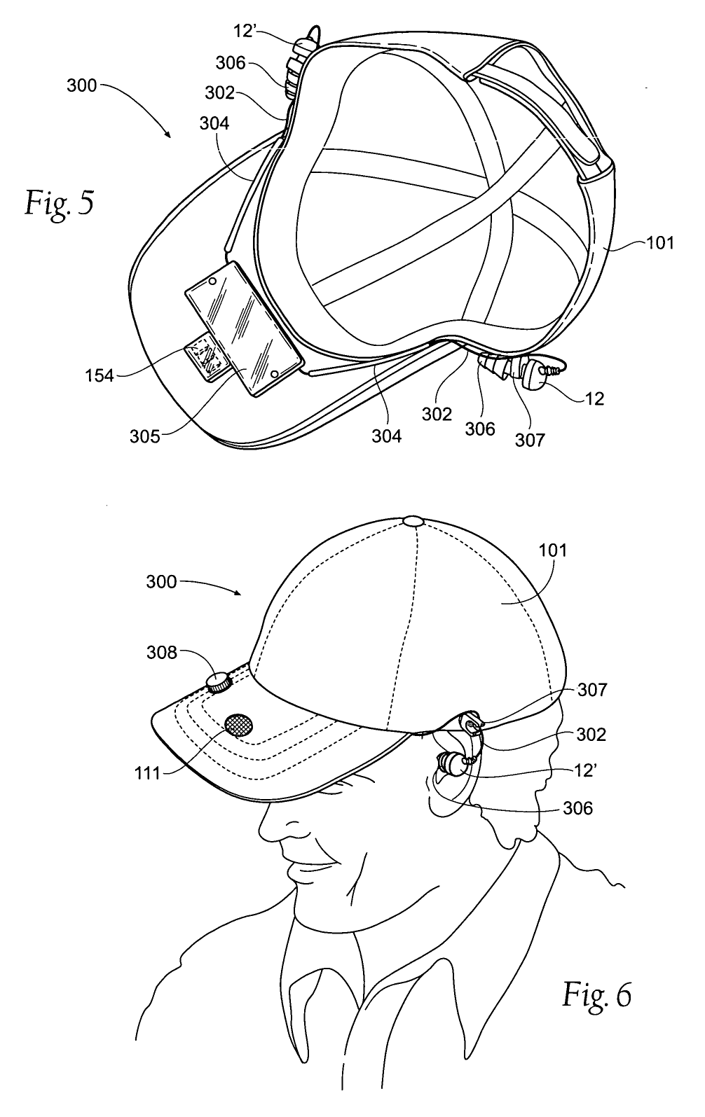

Instead of using tubes to convey sound from a distal speaker as in the previous embodiments, the speakers 12,12′ may generate the sound proximate the ears. Referring to FIGS. 5-6, an embodiment 300 of a hearing protection device of the present invention is shown, wherein the speakers 12,12′ are placed proximate the ears of the wearer. In this embodiment, a housing 305 contains the preferred electrical circuit 10 with the exception of the speakers 12,12′ which are mounted away from the housing 305 but in electrical communication with the rest of the circuit 10 via electrical wires 302. The electrical wires 302 are preferably supported against the hat 101 with, for example, supporting tubular members 304. The speakers 12,12′ are coupled also with the earpieces 306. Retainer clips 307 are attached to the hat 101 to provide anchor points for the speakers 12,12′ and the earpieces 306.

To use the device 300, a user places the hat 101 on his or her head, removes the speakers 12,12′ and earpieces 306 from the retainer clips 307 and places the earpieces 306 in his or her respective ears and powers the device on.

One advantage of the present invention is that typical in-the-ear and behind-the-ear units that normally utilize small batteries can be employed. These are typically zinc-air batteries that start discharging as soon as they are installed. Preferably, a larger module can also be provided, using typical watch batteries (or larger) for much longer periods of intermittent use.

Another advantage of the present invention is that the device may be coupled with a desirable type of hat. Therefore, for use in a warm environment, the device may be coupled with a ventilated cap; for use in a cold environment, the device may be coupled with an insulated cap. Furthermore, as employed in certain embodiments, the present invention disrupts neither the shouldering of a firearm nor the wearing of shooting or safety glasses.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

1. A hearing protection device comprising:
an amplifier having a signal input, a signal output and an associated gain;
a microphone electrically coupled and providing a first electric signal to a compressor circuit, the compressor circuit having an output electrically coupled to the amplifier signal input;
a speaker having an input electrically coupled to the amplifier signal output;
a means for interrupting a second electric signal presented to the speaker input;
a monitor having an input electrically coupled to the amplifier signal input, and further having an output electrically coupled to the means for interrupting; and
an earpiece aurally coupled to the speaker,
wherein all components are carried on a cap worn by a person and the earpiece is selectively removable from a clip on the cap.

2. A hearing protection device comprising:
an amplifier having a signal input, a signal output and an associated gain;
an audio pick-up electrically coupled and providing a first electric signal to the amplifier signal input;
a speaker having an input electrically coupled to the amplifier signal output;
a means for modifying a second electric signal presented to the speaker input;
a monitor having an input electrically coupled to the amplifier signal input, and further having an output electrically coupled to the means for modifying; and
an earpiece aurally coupled to the speaker.

3. A hearing protection device according to claim 2 wherein the means for modifying comprises a means for interrupting the second electric signal.

4. A hearing protection device according to claim 2 wherein the means for modifying comprises a means for modifying the first electric signal, thereby modifying the second electric signal.

5. A hearing protection device according to claim 2 wherein the means for modifying an electric signal presented to the speaker input comprises a means for modifying the amplifier gain.

6. A hearing protection device according to claim 2 further comprising a volume controller, wherein the amplifier further includes a first control input which is electrically coupled to the volume controller.

7. A hearing protection device according to claim 2, wherein the means for modifying comprises a shutdown signal input on the amplifier.

8. A hearing protection device according to claim 2, wherein the audio pick-up is electrically coupled to the amplifier signal input through a compressor circuit.

9. A hearing protection device according to claim 2, wherein the earpiece is aurally coupled to the speaker by a first length of tubing.

10. A hearing protection device according to claim 2, wherein the earpiece is selectively engageable with a human ear.

11. A hearing protection device according to claim 2, the device further comprising a second earpiece aurally coupled to the speaker.

12. A hearing protection device according to claim 2, the amplifier further comprising a second signal output and the device further comprising a second speaker coupled to the amplifier second signal output.

13. A hearing protection device according to claim 12, the device further comprising a second earpiece aurally coupled to the second speaker.

14. A hearing protection device according to claim 2, wherein the audio pick-up and the speaker are carried by a hat.

15. A hearing protection device according to claim 2, said device further comprising a housing, wherein the housing is adapted to at least partially contain the amplifier, the monitor, the audio pick-up and the speaker.

16. In combination with a hat, a hearing protection device comprising:
an amplifier having a signal input, a signal output and an associated gain;
an audio pick-up electrically coupled and providing a first electric signal to the amplifier signal input;
a speaker having an input electrically coupled to the amplifier signal output;
a means for modifying a second electric signal presented to the speaker input;
a monitor having an input electrically coupled to the amplifier signal input, and further having an output electrically coupled to the means for modifying; and
an earpiece aurally coupled to the speaker.

17. A combination according to claim 16, wherein the hat further comprises a brim.

18. A combination according to claim 17, wherein the hearing protector earpiece is selectively attachable to and detachable from the hat.

19. A method comprising the steps of:
(a) detecting a sound and producing a representative electric signal;
(b) monitoring the electric signal;
(c) determining whether the electric signal is above a threshold level;
(d) while the electric signal is above a threshold level, preventing amplification of the electric signal;
(e) preventing amplification of the electric signal for a predetermined period of time; and,
(f) amplifying the electric signal.

20. A method according to claim 19 wherein the predetermined period of time is not more than one second.

21. A method comprising the steps of:
(a) detecting a sound and producing a representative electric signal;
(b) monitoring the electric signal;
(c) amplifying the electric signal by a first gain;
(d) determining whether the electric signal is above a threshold level; and,
(e) while the electric signal is above a threshold level, amplifying the electric signal by a second gain.

22. A method according to claim 21 wherein the second gain is less than the first gain.

23 A hearing protection device comprising:
an amplifier having a signal input, a signal output and an associated gain;
a microphone electrically coupled and providing a first electric signal to a compressor circuit, the compressor circuit having an output electrically coupled to the amplifier signal input;
a speaker having an input electrically coupled to the amplifier signal output;
a means for interrupting a second electric signal presented to the speaker input;
a monitor having an input electrically coupled to the compressor circuit output, and further having an output electrically coupled to the means for interrupting; and
an earpiece aurally coupled to the speaker.
